(19) United States
(12) Patent Application Publication
(10) Pub. No.: US 2018/0168965 A1
(43) Pub. Date: Jun. 21, 2018

(54) Whitening Dentifrice Compositions with Zinc Core Shell Silica Particles

(71) Applicant: Colgate-Palmolive Company, New York, NY (US)

(72) Inventors: Agnieszka Kocinska, Basel (CH); Jennifer GRONLUND, Flemington, NJ (US); Hallena STROTMAN, Somerset, NJ (US); Suman CHOPRA, Monroe, NJ (US)

(73) Assignee: Colgate-Palmolive Company, New York, NY (US)

(21) Appl. No.: 15/846,944

(22) Filed: Dec. 19, 2017

Related U.S. Application Data
(60) Provisional application No. 62/437,111, filed on Dec. 21, 2016.

(51) Int. Cl. A61K 8/27; A61Q 11/00; A61K 8/25; A61K 8/02

(52) U.S. Cl. CPC A61K 8/27; A61Q 11/00; A61K 2800/30; A61K 8/0241; A61K 2800/434; A61K 8/25

(57) ABSTRACT

A whitening dentifrice composition, free from peroxide whitening agents, includes a blue coloring agent, a zinc core shell silica (Zn-CSS) particle, and an orally acceptable vehicle including a non-aqueous solvent and water. The blue coloring agent includes at least one of a blue pigment and a blue dye and has a blue to blue-violet color with a hue angle in the CIELAB system ranging from 200 degrees to 320 degrees. The Zn-CSS particle includes a silica core, and a surface of the silica core etched with a metal silicate, which is a silicate of zinc ion and optionally a monovalent metal ion.

### BACKGROUND

The natural off-white or yellow color of teeth can be made to appear whiter through the use of tooth whitening products. Typically, the whitening effect of the commonly available tooth whitening products is either achieved by chemically altering or removing the stain, such as by use of peroxides and/or changing the visual perception of the color of the teeth. It is known in the literature that the visual perception of a white substance can be altered through the deposition of an optical brightener, or a blue coloring agent on to teeth. Blue coloring agents, such as blue pigments or blue dyes having the hue angle (in the CIELAB scale) of the reflected or emitted light between 200 to 320 degrees are most useful in making the natural off-white or yellow color of teeth appear whiter through the deposition onto teeth. Pigments have been used with a deposition aid, such as Gantrez® (copolymers of maleic anhydride and with methyl vinylether) in toothpaste to make teeth look whiter, as disclosed in EP19353958A1. However, dyes are much more difficult to deposit onto teeth and be retained in the hydrated environment of the oral cavity due to dyes being much more soluble in water than pigments. U.S. Pat. No. 6,030,222 discloses depositing dyes on teeth when blended with specific carriers. US Patent Application Publication 2012/0093905 discloses dyes coupled to certain polymers.

However, there is still need for new dentifrice compositions and new methods of increasing whitening efficacies of both dyes and pigments, irrespective of the dentifrice compositions.

### BRIEF SUMMARY

Disclosed herein is a whitening dentifrice composition comprising:

a blue coloring agent comprising at least one of a blue pigment and a blue dye, wherein the blue coloring agent has a blue to blue-violet color with a hue angle in the CIELAB system ranging from 200 degrees to 320 degrees:

a zinc core shell silica (Zn-CSS) particle, wherein the Zn-CSS particle comprises a silica core, and a surface of the silica core etched with a metal silicate, wherein the metal silicate is a silicate of zinc ion and optionally a monovalent metal ion; and

an orally acceptable vehicle comprising a non-aqueous solvent and water,

wherein the whitening dentifrice composition is free from peroxide whitening agents.

In an embodiment of the whitening dentifrice composition, the zinc ion is present in an amount of from 0.01 to 1 weight %, based on the total amount of the whitening dentifrice composition.

In another embodiment of the whitening dentifrice composition, the surface of the silica core of the Zn-CSS particle is represented by the following formula:

(SiO₂)ₚ[Oₒ*Mₙ⁺Znₘ²⁺Hₕ⁺].qH₂O

wherein O* is oxygen in the silicate form; M is a monovalent metal ion; Zn is divalent zinc ion; p, o, n, m, h and q are the atomic percentages of each component; and the total charge of each core shell silica particle is zero.

In one embodiment of the whitening dentifrice composition, the non-aqueous solvent is present in an amount from 25 to 95 weight % and water is present in an amount from 3 to 30 weight %, based on the total amount of the whitening dentifrice composition.

In another embodiment of the whitening dentifrice composition, the non-aqueous solvent is in present in an amount from 3 to 50 weight % and water is present in an amount from 25 to 70 weight %, based on the total amount of the whitening dentifrice composition.

In yet another embodiment of the whitening dentifrice composition, the blue coloring agent is a blue dye present in an amount of from 0.02 to 2 weight° %, based on the total amount of the whitening dentifrice composition. In a specific example, the blue coloring agent comprises at least one of FD&C Blue#1, FD&C Blue #2, D&C Blue #4, CI Food Blue 5, and Acid Blue 1.

In one embodiment of the whitening dentifrice composition, the blue coloring agent is a blue pigment present in an amount of from 0.01 to 0.075 weight %, based on the total amount of the whitening dentifrice composition. The blue coloring agent comprises at least one of a violet pigment #1 through to violet pigment #56 and pigment blue 1 through to pigment blue 83, as listed in the Color Index International. In a specific example, the blue coloring agent is a blue pigment #15 or a blue pigment #5.

One object of the whitening dentifrice composition is to provide a greater whitening benefit for the blue coloring agent than a comparative dentifrice composition that is identical to the whitening dentifrice composition as disclosed hereinabove, except that the comparative dentifrice composition either has core shell silica with no zinc (CSS) or has no core shell silica. The greater whitening benefit comprises at least one of greater initial whitening benefit and a longer-lasting whitening benefit.

In an embodiment of the whitening dentifrice composition, the non-aqueous solvent comprises glycerin, sorbitol, xylitol, propylene glycol, polyols, ketones, aldehydes, carboxylic acids or salts thereof, amines, or mixtures thereof.

In an aspect, there is a method for whitening a tooth surface comprising contacting the whitening dentifrice composition as described herein with the tooth surface.

In another aspect, there is a use of a zinc core shell silica (Zn-CSS) particle as a tooth whitening enhancing agent in a whitening dentifrice composition, wherein the whitening dentifrice composition further comprises:

a blue coloring agent comprising at least one of a blue pigment and a blue dye, wherein the blue coloring agent has a blue to blue-violet color with a hue angle in the CIELAB system ranging from 200 degrees to 320 degrees; and

an orally acceptable vehicle comprising a non-aqueous solvent and water,

wherein the Zn-CSS particle comprises a silica core, and a surface of the silica core etched with a metal silicate, wherein the metal silicate is a silicate of zinc ion or zinc ion and a monovalent metal ion, and

wherein the whitening dentifrice composition is free from peroxide whitening agents.

Further areas of applicability of the present invention will become apparent from the detailed description provided hereinafter. It should be understood that the detailed description and specific examples, while indicating some preferred aspects of the invention, are intended for purposes of illustration only and are not intended to limit the scope of the invention.

### DETAILED DESCRIPTION

The following description of various preferred aspect(s) is merely exemplary in nature and is in no way intended to limit the invention, its application, or uses.

As used throughout, ranges are used as shorthand for describing each and every value that is within the range as well as the endpoints. Any value within the range can be selected as the terminus of the range. In addition, all references cited herein are hereby incorporated by reference in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

As used herein, the phrase “Core Shell Silica” refers to a core shell silica particle comprising a silica core, and a surface of the silica core etched with a metal silicate, wherein the metal silicate is a silicate of a monovalent metal ion of the formula: M¹₂SiO_{3.X} H₂O, wherein M¹ is a monovalent metal ion, optionally a group I metal ion, and x is from 0 to 10. M¹ is preferably Na⁺ or K⁺.

As used herein, the phrase “Zinc Core Shell Silica (Zn-CSS)” refers to a core shell silica particle comprising a silica core, and a surface of the silica core etched with a metal silicate, wherein the metal silicate is a silicate of zinc ion and optionally a monovalent metal ion. Hence, the metal silicate may be zinc silicate or a mixture of zinc silicate and a monovalent metal silicate.

As used herein, the “surface of the silica core” can be an outer surface of the silica core or an internal surface of the silica core.

As used herein, the term “d(0.5)” is used interchangeably with “D(0.5)” or “d50” and refers to the diameter (typically in microns) of the particles that splits the distribution with half the population above and half below this diameter. The Dv50 (or Dv0.5) is the median for a volume distribution, Dn50 is used for number distributions, and Ds50 is used for surface distributions. In the present context, d(0.5) will be used to refer to the median particle size for a volume distribution (Dv0.5). Similarly, the d(0.1) value of the particles is the diameter that splits the distribution with 10/o of the population below and 90% above this diameter and the d(0.9) value of the particles is the diameter that splits the distribution with 90° % of the population below and 10% above this diameter.

As used herein, the term “span” refers to the distribution width of the particle size distribution and is calculated as follows:

Span=(d(0.9)−d(0.1))/d(0.5)

As used herein, an “orally acceptable vehicle” refers to a material or combination of materials that are safe for use in the whitening dentifrice compositions of the present disclosure, commensurate with a reasonable benefit/risk ratio, with which the whitening agent, and other desired active ingredients may be associated while retaining significant efficacy.

As used herein, the phrase “whitening efficacy” refers to the amount of change in tooth color. The color change may be measured according to the L*a*b* color scale, such as “CIELAB” color measurement system or standard adopted by the Commission Internationale de I'Eclairage (CIE) in 1976. It is based on a three-dimensional CIELAB color space. The system was developed to represent color in a manner that is consistent with human vision and proportional to perceived color differences. CIELAB values describe coordinates of a specific color in the three dimensional CIELAB color space. There are three axes: L* (defining light to dark); b* (defining blue to yellow); and a* (defining red to green). Any point in the three dimensional CIELAB color space may be defined by its L*, a*, and b* coordinates. The same point may also be defined by L*, hue angle, and chroma, which uses cylindrical coordinates. The hue angle is defined by the formula: H_{ab}=tan⁻¹ (b*/a*), where a* and b* are coordinates in the L*a*b* three dimensional CIELAB color space. A detailed description of hue angle may be found in M. L. Gulrajani (Ed.), (2010). Colour Measurement: Principles, Advances and Industrial Applications. Cambridge, United Kingdom: Woodhouse Publishing, which is herein incorporated by reference in its entirety.

The luminance or lightness (L*) value measures brightness and varies from a value of one hundred for perfect white to zero for black, assuming a* and b* are zero. The a* value is a measure of redness when positive, gray when zero and greenness when negative. The b* value is a measure of yellowness when positive, gray when zero and blueness when negative. Generally, teeth appear whiter as: the L* value increases meaning they become brighter; the a* value increases or decreases, depending upon whether the stained teeth have a green tint or red tint prior to whitening; and the b* value decreases meaning they become less yellow. While this is the general relationship for perceived whitening, the b* value might also slightly increase if the magnitude of the increase of the L* value is large enough. Similarly, the L* value might also decrease if the magnitude of the decrease of the b* value is large enough to overshadow the less significant change in L*.

As used herein, the phrase “whiteness index” is based on the distance of a color value from a nominal white point, represented in the CIELAB color space as L*=100, a*=0 and b*=0, and is defined according to formula (1).

WIO=[(a*)²+(b*)²+(L*−100)²]/2 (1)

Changes in the whiteness index may be used to assess the whitening efficacy (ΔWIO) of a composition before and after a treatment. The whitening efficacy (ΔWIO) may be calculated according to formula (2):

ΔWIO=WIO(Treatment)−WIO(baseline) (2)

Other values which may be used to assess tooth whiteness are described in Joiner et al., “A Review of Tooth Color and Whiteness”, Journal of Dentistry, 2008, 36S:S2-S7, which is incorporated herein by reference in its entirety.

The present inventors have surprisingly discovered that zinc core shell silica (Zn-CSS) particles when included in a whitening dentifrice composition, which also includes a blue coloring agent as described hereinbelow, enhances whitening efficacy, as shown by at least one of greater initial whitening benefit and a longer-lasting whitening benefit. Additionally, due to the enhanced whitening efficacy, the whitening dentifrice compositions may be formulated with reduced amounts of blue coloring agents without a concomitant decrease in whitening efficacy. Accordingly, the present whitening dentifrice compositions exhibit improved properties in comparison to art-known whitening compositions.

### Compositions

In an aspect, there is a whitening dentifrice composition that includes a blue coloring agent including a blue pigment and/or a blue dye, wherein the blue coloring agent has a blue to blue-violet color with a hue angle in the CIELAB color measurement system ranging from 200 degrees to 320 degrees; a zinc core shell silica (Zn-CSS) particle, wherein the Zn-CSS particle comprises a silica core, and a surface of the silica core etched with a metal silicate, wherein the metal silicate is a silicate of zinc ion or a mixture of silicate of zinc ion and a silicate of monovalent metal ion; and an orally acceptable vehicle that includes a non-aqueous solvent and water, wherein the whitening dentifrice composition is free from peroxide whitening agents.

By “free from peroxide whitening agents”, it is meant that the composition contains less than 0.1 weight %, less than 0.05 weight %, or less than 0.01% peroxide whitening agents; or substantially no peroxide whitening agents; or that the composition contains no peroxide whitening agents. In certain embodiments, the composition contains no peroxide whitening agents. The tooth whitening effect of the whitening dentifrice compositions of the present disclosure is provided by the presence of the dye or pigment, rather than by the presence of any peroxide whitening agents.

In an aspect, the whitening dentifrice composition of the present disclosure may further include an abrasive, a surfactant, a foaming agent, a vitamin, a fluoride ion source, an adhesive material, an enzyme, a humectant, a thickener, a sweetener, an anticalculus agent, a pH modifier, an antimicrobial agent, a preservative, and/or a flavoring.

### Zinc Core Shell Silica (Zn-CSS) Particles

As disclosed hereinabove, the Zn-CSS particles of the present disclosure comprise a silica core, and a surface of the silica core etched with a metal silicate, wherein the metal silicate is a silicate of zinc ion or a mixture of silicates of zinc ion and a monovalent metal ion. The Zn-CSS particles may be present in an amount ranging from 10 to 30 weight %, or 15 to 30 weight %, or 15 to 25 weight %, based on the total amount of the whitening dentifrice composition. In another aspect, the zinc ion is present in an amount of from 0.1 to 5 weight %, 0.15 to 3 weight/o, or 0.15 to 1.5 weight %, or 0.2 to 1 weight %, based on the total amount of the whitening dentifrice composition. In an embodiment, the zinc ion is present in an amount of less than 1 weight %, based on the total amount of the whitening dentifrice composition. In another embodiment, Zn-CSS particles may be present in an amount of 22 weight % delivering 1 weight % or less of zinc ion, based on the total amount of the whitening dentifrice composition.

The Zn-CSS particles of the present disclosure typically comprise a plurality of monolayers of metal silicate. The number of monolayers may be from 2 to 100, 2 to 40, 2 to 12 or 12 to 40 layers. The Zn-CSS particles may comprise 2, 4, 16, 32, 36, or anywhere between 2 and 36 monolayers.

By way of example, the Zn-CSS particles can be prepared by:

i) admixing an amount of silica particles in water with an amount of a base, wherein the base comprises a monovalent metal ion, to produce core shell silica particles, each core shell silica particle comprising a silica core, and a surface of the silica core etched with a silicate of the monovalent metal ion; and

ii) reacting the core shell silica particles formed in step i) with a zinc salt such as zinc chloride, to form zinc core shell silica particles comprising zinc silicate of the formula, ZnSiO₃.xH₂O, wherein x is from 0 to 10, on the surface of the silica core.

The silica particles may be selected from among precipitated silica, fumed silica and fused silica particles. The monovalent ion may be Na⁺ and/or K⁺.

The zinc salt may be selected from among zinc acetate, zinc borate, zinc butyrate, zinc carbonate, zinc chloride, zinc citrate, zinc formate, zinc gluconate, zinc glycerate, zinc glycolate, zinc lactate, zinc oxide, zinc phosphate, zinc picolinate, zinc proprionate, zinc salicylate, zinc silicate, zinc stearate, zinc tartrate, zinc undecylenate and mixtures thereof. In one embodiment, the zinc salt is zinc chloride.

In an aspect, the surface of the silica core is the outer surface of the silica core. In addition or as an alternative, the surface of the silica core may be an internal surface of the silica core.

The surface of the silica core of the Zn-CSS particle may have a depth of 10 nm and may comprise from 0.1 to 15 weight % of zinc and/or metal ion, based on the total amount of the Zn-CSS particle. In one embodiment, the surface of the silica core of the Zn-CSS particle is represented by the following formula:

(SiO₂)ₚ[Oₒ*Mₙ⁺Znₘ²⁺Hₕ⁺].qH₂O

wherein O* is oxygen in the silicate form; M is a monovalent metal ion; Zn is divalent zinc ion; p, o, n, m, h and q are the atomic percentages of each component; and the total charge of each core shell silica particle is zero.

The atomic percentage for each component except H⁺ can be typically determined by electron spectroscopy for chemical analysis (ESCA).

The Zn-CSS particles may comprise up to 15 weight % of total metal, preferably zinc, based on the total amount of the Zn-CSS particles. In an embodiment, zinc ion may be present on a Zn-CSS particle in an amount of 0.1 to 15 weight % or 4 to 14.5 weight % or 8 to 14.5 weight %, based in the total amount of the Zn-CSS particle.

In an aspect, the amount of zinc adsorbed to surface monolayers of the of Zn-CSS particles is less than 500% or in the range of 30-35% or 33% of the maximum ion-exchange capacity of the particle for divalent ions.

The d(0.5) value of the Zn-CSS particles is typically from 5 nm to 50 μm.

The d(0.5) value of the Zn-CSS particles may be from 26 μm to 40 μm or from 18 μm to 25 μm or from 10 μm to 15 μm. Particles having a d(0.5) value within this range are typically opaque. Translucent particles are those which allow light to pass through, although it is not possible to see an image through the particles. This is distinguished from transparent compositions which allow light to pass through and an image can be seen through the composition. Methods for determining particle size are well known in the art. For example, particle size may be determined using light scattering methodologies, such as using the Mastersizer 2000, Hydro 2000S, Malvern Instruments Limited.

In an aspect, the d(0.5) value of the Zn-CSS particles may be from 5 μm to 15 μm or from 2.5 μm to 4.5 μm.

In another aspect, the d(0.5) value of the Zn-CSS particles may be from 5 nm to 20 nm, or from 10 nm to 15 nm, or from 5 nm to 12 nm.

In an aspect, the span of the Zn-CSS particles according to the present disclosure is typically from 1.5 to 3.

In a preferred embodiment, the Zn-CSS particles have a d(0.1) of from 10 to 13 μm, a d(0.5) of from 30 to 33 μm, and a d(0.9) of from 61 to 64 μm.

In another preferred embodiment, the Zn-CSS particles have a d(0.1) of from 6 to 9 μm, a d(0.5) of from 18 to 21 μm, and a d(0.9) of from 41 to 45 μm.

In a further preferred embodiment, the Zn-CSS particles have a d(0.1) of from 3 to 5 μm, a d(0.5) of from 11 to 14 μm, and a d(0.9) of from 33 to 36 μm.

In preferred embodiments, the d(0.5) value of the Zn-CSS particles is less than the mean diameter of a human dentin tubule. This allows the Zn-CSS particles to enter the dentin tubules, which may be exposed on damage to the protective enamel layer. In human teeth, the mean diameter of dentin tubules near the dentino-enamel junction is 0.9 μm, the middle section of the dentin tubule has a mean diameter of 1.2 μm, and near the pulp the mean diameter is 2.5 μm.

In another aspect, a silica source is selected to produce Zn-CSS particles which fit into the dentin tubule (e.g. Aerosil® 200—a fumed silica (synthetic amorphous silica) with a d(0.5) of 0.012 μm). In another embodiment of the invention, the d(0.5) value of the Zn-CSS particles is less than 0.9 μm. In still another embodiment of the invention, the Zn-CSS particle has a d(0.5) in the range of 0.010 μm to less than 0.9 μm. In another aspect, the Zn-CSS particles can also plug, block holes in the enamel.

In an aspect, the Zn-CSS particles have a surface charge density of from 0.5 to 4.5 meq/g silica or from 2 to 3 meq/g silica or from 2.45 to 2.55 meq/g silica.

In an aspect, the core shell silica particles have a charge, or ion-exchange capacity of, from 0.05 to 0.1 C/cm² surface area, or from 0.085 to 0.095 C/cm2 surface area or from 0.089 C/cm² surface area.

### Blue Coloring Agent

As used herein, the term “blue coloring agent” refers to a substance in the form of a dry powder or liquid that imparts color to another substance. Generally, coloring agents include pigments, dyes, lakes, or combinations thereof.

In an aspect, the blue coloring agent has a blue to blue-violet color with a hue angle in the CIELAB system ranging from 200 degrees to 320 degrees.

### Blue Coloring Agent—Pigments

In some embodiments, the whitening dentifrice compositions of the present disclosure may include a pigment. As used herein, a “pigment” is a synthetic or natural water insoluble substance, which imparts color to another substance. In some embodiments, the pigments further enhance the whiteness of the teeth. As is known in the art, the visual perception of a white substance can be altered through the deposition of an optical brightener, a blue pigment, or a blue dye. This effect is commonly used in laundry detergent products to make white clothes appear “whiter” to the human eye. The same concept has been applied to tooth whitening. See PCT Publication No. WO 2015/099642 to Colgate-Palmolive Company, which is herein incorporated by reference in its entirety.

In some embodiments, the pigment included in the whitening dentifrice compositions of the present disclosure may have a hue angle, h, in the CIELAB system ranging from 220 degrees to 320 degrees, typically between 250 degrees and 290 degrees.

The pigment used in the whitening dentifrice compositions is capable of reflecting sufficient light such that the treated tooth is perceivably whiter than its initial color. In some embodiments, the pigment may be colored such that its natural color is within the violet-red to green-blue color. More particularly, the pigment may be violet or blue, e.g., one of those listed in the Color Index International. These pigments are listed as violet pigment #1 through to #56 and blue pigment #1 through #83. In some embodiments, the violet pigment may be violet pigment #1, 1:1, 1:2, 2, 3, 5:1, 13, 19, 23, 25, 27, 31, 32, 37, 39, 42, 44 and/or 50. In some embodiments, the blue pigments may be blue pigment #1, 2, 9, 10, 14, 15, 15:1, 15:2, 15:3, 15:4, 15:6 16, 18, 19, 24:1, 25, 56, 60, 61, 62 and/or 66. Other suitable pigments are pigment ultramarine blue and ultramarine violet. Typically, the pigment is blue pigment #15, more typically blue pigment #15:1, 15:2, 15:3, 15:4, 15:5 or 15:6, most typically 15:1.

While blue or violet single pigments may be used in the whitening dentifrice compositions, the same effect may be achieved through mixing pigments outside of the hue angle range of 220 degrees to 320 degrees. The desired hue angle may instead be obtained by mixing a red and green-blue pigment to yield a blue or violet shaded pigment.

The amount of pigment in the whitening dentifrice composition may be from 0.01 to 0.075 weight %, such as 0.05%. In other embodiments, the amount of pigment in the whitening dentifrice composition may be from 0.01 to 0.05 weight %, or from 0.03 to 0.05%, by weight based on the total amount of the whitening dentifrice composition. The pigment may be uniformly spread throughout the whitening dentifrice composition or may be dispersed in a second phase such as a stripe or other coextruded second phase. Such “dual phase” compositions have the advantage that the phases may be differently colored, presenting a more visually attractive product to the consumer.

### Blue Coloring Agent—Dyes

As used herein, the term “dye” refers to an organic species, which is essentially water soluble in an aqueous medium in which the dye remains chemically stable. The dyes used with the whitening dentifrice composition of the present disclosure are generally food color additives presently certified under the Food Drug & Cosmetic Act for use in food and ingested drugs, including dyes such as FD&C Red No. 3 (sodium salt of tetraiodofluorescein), FD&C Yellow No. 5 (sodium salt of 4-p-sulfophenylazo-1-p-sulfophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-N-ethyl-N-p-sulfobenzyl)-.DELTA.-3,5-cyclohexadienimine], FD&C Blue No. 1 (disodium salt of dibenzyldiethyl-diaminotriphenylcarbinol trisulfonic acid anhydride), FD&C Blue No. 2 (sodium salt of disulfonic acid of indigotin) D&C Green No. 5, D&C Orange No. 5, D&C Red No. 21, D&C Red No. 22, D&C Red No. 27, D&C Red No. 28, D&C Red No. 30, D&C Red No. 40, D&C Yellow No. 10 and mixtures thereof in various proportions.

In an aspect, the blue coloring agent is a blue dye selected from among FD&C Blue#1, FD&C Blue #2, D&C Blue #4, CI Food Blue 5, Acid Blue 1, or a mixture thereof.

The amount of one or more of the dyes in the oral care composition may widely vary. For example, the amount of one or more of the dyes in the whitening dentifrice composition of the present disclosure may be from 0.02 to 2 weight %, or 0.02 to 1.5 weight %, or 0.02 to 1 weight %, or 0.02 to 0.5 weight %, 0.02 to 0.15 weight %, or 0.02 to 0.1 weight %, based on the total amount of the whitening dentifrice composition. In at least one embodiment, the one or more dyes may be disposed or dispersed uniformly throughout the whitening dentifrice composition. In another embodiment, the one or more dyes may be disposed or dispersed in different phases of the whitening dentifrice composition. For example, one or more of the dyes may be disposed or dispersed in a first phase (e.g., a hydrophobic phase) of the whitening dentifrice composition, and one or more of the remaining dyes, or no dye, may be disposed or dispersed in a second phase (e.g., a hydrophilic phase) of the whitening dentifrice composition.

### Orally Acceptable Vehicles

In an aspect, a blue coloring agent, such as a blue pigment or a blue dye and zinc core shell silica particles, are combined with an orally acceptable vehicle to form a whitening dentifrice composition. Such dentifrices may include a dental tablet, toothpaste (e.g., dental cream), tooth powders, a viscous liquid, such as a gel, a mouth wash, or any other form known to one of skill in the art. For example, the dentifrice may be a gel.

The orally acceptable vehicle may comprise a non-aqueous solvent and water.

The orally acceptable vehicle may be present in an amount of from 25 to 99 weight %, or 35 to 85 weight %, or 40 to 80 weight %, or 60 to 80 weight %, with water present in an amount of 3 to 50 weight %, 10 to 40 weight %, 15 to 35 weight %, based on the total amount of the whitening dentifrice composition of the present disclosure.

It has been surprisingly discovered that Zn-CSS particles can be extremely effective at maintaining increased tooth whitening when the dye is incorporated into a low water content orally acceptable vehicle.

The non-aqueous solvent for the orally acceptable vehicles may include alcohols, polyhydric alcohols such as glycerin, sorbitol, xylitol, propylene glycol, polyols, ketones, aldehydes, carboxylic acids or salts thereof, amines, or mixtures thereof. Such materials typically also function as humectants.

In an aspect, the orally acceptable vehicle is a low-water content orally acceptable vehicle and may include any known ingredients or additives. For example, the orally acceptable vehicle may include a liquid mixture of water, glycerin, and sorbitol. The low-water content orally acceptable vehicle may comprise non-aqueous solvent in the range of from 25 to 95 weight %, or 25 to 75 weight %, or 25 to 55 weight % and water in the range of from 3 to 25 weight %, or 3 to 20 weight %, or 3 to 15 weight %, based on the total amount of the whitening dentifrice composition.

In another aspect, the orally acceptable vehicle is a high-water content orally acceptable vehicle and may include any known ingredients or additives, such as for example, a mixture of water, glycerin, and sorbitol. The high-water content orally acceptable vehicle may include non-aqueous solvent in the range of from 3 to 50 weight %, or 5 to 40 weight %, or 5 to 30 weight % and water in the range of from 25 to 70 weight %, or 25 to 50 weight %, or 25 to 45 weight %, based on the total amount of the whitening dentifrice composition.

In an aspect, where the orally acceptable vehicle has low-water content, the orally acceptable vehicle may include an ethylene oxide, propylene oxide block co-polymer, such as one having the formula (ethylene oxide)ₓ-(propylene oxide)_{y}, wherein x is an integer of 80-150, e.g., 100-130, e.g., 118, and y is an integer of 30-80, e.g., 60-70, e.g., 66, having an average molecular weight of greater than 5000, e.g., 8000-13000 Da, e.g., 9800. In some embodiments, the ethylene oxide, propylene oxide co-polymer is substantially free of ethylene oxide, propylene oxide block co-polymer of average molecular weight less than 5000 Da. An example of a suitable commercially available ethylene oxide, propylene oxide co-polymer is PLURACARE® L1220 (available from BASF, Wyandotte, Mich., United States of America).

Other suitable orally acceptable vehicles, which may be incorporated into the whitening dentifrice composition having low-water content may include, for example polyethylene glycol, such as PEG400, PEG600, PEG/PPG copolymers, such as PEG/PPG 38/8 copolymer, and PEG/PPG-1 16/66 copolymer sold as PLURACARE® L4370 and PLURACARE® L1220 from BASF, Wyandotte, Mich., respectively. In some embodiments, polyethylene glycol may be present in the whitening dentifrice compositions in an amount ranging from 0 to 0.01 weight %, 0.01 to 60 weight %, or 0 to 15 weight %, such as 10 weight %, 6.3 weight %, 7.5 weight %, or 15 weight %, based on the total amount of the whitening dentifrice composition.

In some embodiments, the orally acceptable vehicle further includes a polymer, which aids in the deposition of a pigment, such as blue pigment (“pigment deposition aid”). Without wishing to be bound by theory, it is believed that a pigment deposition aid works by having affinity for both the pigment and the surface of the teeth, the deposition aid serving as a link between the two.

Pigment deposition aids for use in accordance with the present disclosure may be or include high molecular weight polymers, i.e. polymers having a molecular weight of 200,000 or greater. Suitable pigment deposition aids include hydroxypropylmethylcellulose polymers, for example, hydroxypropylmethylcellulose polymers having a viscosity (mPa-s) at 20° C. of 3.200 to 4.800 using capillary viscometry. Suitable commercially available hydroxypropylmethylcellulose polymers include WALOCEL® HM 4000PA-2910 from Covestro AG, Leverkusen, Germany. Other suitable pigment deposition aids include those described in European Patent No. 1935395, such as GANTREZ™ polymers, available from Ashland, Inc., Wilmington, Del., which is herein incorporated by reference in its entirety.

The pigment deposition aid is incorporated into the instant whitening dentifrice compositions in an amount from 0 weight %, 0.01 to 10 weight %, 0.05 to 5 weight %, or 0.1 to 1 weight %, by weight.

In one embodiment, the pigment deposition aid is able to enhance the whitening efficacy of the dentifrice composition in comparison to a dentifrice composition that does contain a pigment deposition aid, by at least 5% and more typically by at least 25%.

In some embodiments, the viscosity of the whitening dentifrice compositions of the present disclosure may be from 1,000 centipoise (cPs) to 900,000 cPs, such as 10,000 cPs to 100,000 cPs, such as 50,000 cPs to 900,000 cPs, such as 200,000 cPs to 600,000 cPs.

### Surfactants

In an aspect, the whitening dentifrice composition may further include a surfactant. In some embodiments, the surfactant enhances stability of the formulation, helps clean the oral cavity surfaces through detergency, and provides foam upon agitation, e.g., during brushing with the whitening dentifrice composition of the disclosure. Surfactants generally achieve increased whitening action, by thoroughly dispersing the whitening agent throughout the oral cavity. In various embodiments, suitable surfactants may function as a surface-active agent, emulsifier, and/or foam modulator.

Any orally acceptable surfactant, most of which are anionic, nonionic or amphoteric, can be used. Suitable anionic surfactants include without limitation water-soluble salts of C8-20 alkyl sulfates, sulfonated monoglycerides of C8-20 fatty acids, sarcosinates, taurates and the like. Illustrative examples of these and other classes include sodium lauryl sulfate, sodium cocoyl monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include, without limitation, derivatives of C8-20 aliphatic secondary and tertiary amines having an anionic group such as carboxylate, sulfate, sulfonate, phosphate or phosphonate. A suitable example is cocoamidopropyl betaine.

In some embodiments, one or more surfactants may be present in a total amount of from 1.8 to 4 weight %, or 1.9 to 2 weight %, based on the total amount of the whitening dentifrice composition. In some embodiments, one or more surfactants may be present in a total amount of 2 weight %, based on the total amount of the whitening dentifrice composition.

### Thickener

In some embodiments, the whitening dentifrice composition of the present disclosure, optionally, includes a thickener. Any orally acceptable thickener can be used, including without limitation carbomers, also known as carboxyvinyl polymers, carrageenans, also known as Irish moss and more particularly carrageenan (iota-carrageenan), high molecular weight polyethylene glycols (such as CARBOWAX™, available from The Dow Chemical Company), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, and colloidal and/or fumed silica and mixtures of the same. In some embodiments, the one or more optional thickeners are present in a total amount of 0.1 to 90 weight %, or 1 to 50 weight %, or 5 to 35 weight %, based on the total amount of the whitening dentifrice composition.

### Antioxidants

In some embodiments, the present whitening dentifrice compositions optionally include an antioxidant. Acceptable antioxidants include BHA, BHT, vitamin A, carotenoids, vitamin E, flavonoids, polyphenols, ascorbic acid, herbal antioxidants, chlorophyll, melatonin and mixtures thereof. In some embodiments, the one or more antioxidants are optionally present in a total amount of 0.01 to 0.1 weight %, such as 0.03 weight %, by weight, based on the total amount of the whitening dentifrice composition.

### Flavoring Agents

Useful flavoring agents include any material or mixture of materials operable to enhance the taste of the whitening dentifrice composition. Any orally acceptable natural or synthetic flavoring agent can be used, such as flavoring oils, flavoring aldehydes, esters, alcohols, similar materials, and combinations thereof. Flavoring agents include vanillin, sage, marjoram, parsley oil, spearmint oil, cinnamon oil, oil of wintergreen (methylsalicylate), peppermint oil, clove oil, bay oil, anise oil, eucalyptus oil, citrus oils, fruit oils and essences including those derived from lemon, orange, lime, grapefruit, apricot, banana, grape, apple, strawberry, cherry, pineapple, etc., bean- and nut-derived flavors such as coffee, cocoa, cola, peanut, almond, etc., adsorbed and encapsulated flavorants, and mixtures thereof. Also encompassed within flavoring agents herein are ingredients that provide fragrance and/or other sensory effect in the mouth, including cooling or warming effects. Such ingredients include menthol, menthyl acetate, menthyl lactate, camphor, eucalyptus oil, eucalyptol, anethole, eugenol, cassia, oxanone, x-irisone, propenyl guaiethol, thymol, linalool, benzaldehyde, cinnamaldehyde, N-ethyl-p-menthan-3-carboxamine, N,2,3-trimethyl-2-isopropylbutanamide, 3-1-menthoxypropane-1,2-diol, cinnamaldehyde glycerol acetal (CGA), methone glycerol acetal (MGA) and mixtures thereof.

In some embodiments, one or more flavoring agents are optionally present in a total amount of 0.01 to 5 weight %°, or 0.05 to 2 weight %, or 0.1 to 2.5 weight %, or 0.1 to 0.5 weight %, or 1.4 weight %, based on the total amount of the whitening dentifrice composition.

### Sweeteners

Sweeteners among those useful herein include orally acceptable natural or artificial, nutritive or non-nutritive sweeteners. Such sweeteners include dextrose, polydextrose, sucrose, maltose, dextrin, dried invert sugar, mannose, xylose, ribose, fructose, levulose, galactose, corn syrup (including high fructose corn syrup and corn syrup solids), partially hydrolyzed starch, hydrogenated starch hydrolysate, sorbitol, mannitol, xylitol, maltitol, isomalt, aspartame, neotame, saccharin and salts thereof, sucralose, dipeptide-based intense sweeteners, cyclamates, dihydrochalcones and mixtures thereof. Some embodiments optionally include one or more sweeteners. In some embodiments, the one or more optional sweeteners are present in a total amount from 0.005 to 5% or 0.01 to 1 weight %, based on the total amount of the whitening dentifrice composition.

### pH Modifying Agents

pH modifying agents among those useful herein include acidifying agents to lower pH, basifying agents to raise pH and buffering agents to control pH within a desired range. For example, one or more compounds selected from acidifying, basifying and buffering agents can be included to provide a pH of 2 to 10, or in various embodiments from 2 to 8, from 3 to 9, from 4 to 8, from 5 to 7, from 6 to 10, and from 7 to 9. Any orally acceptable pH modifying agent can be used, including without limitation carboxylic, phosphoric and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole and mixtures thereof. One or more pH modifying agents are optionally present in a total amount effective to maintain the whitening dentifrice composition in an orally acceptable pH range.

### Other Active Ingredients

Colorants, mouth-feel agents and/or others additives may also be included, if desired, in the present whitening dentifrice compositions.

The whitening dentifrice compositions of the present disclosure optionally include one or more further active material(s), which is operable for the prevention or treatment of a condition or disorder of hard or soft tissue of the oral cavity, the prevention or treatment of a physiological disorder or condition, or to provide a cosmetic benefit.

Some embodiments of the present disclosure include a dental abrasive or combination of dental abrasive agents. As used herein, the term “abrasive” or “abrasive agent” also includes materials commonly referred to as “polishing agents.” Any orally acceptable abrasive can be used, but typically, type, fineness (particle size) and amount of abrasive should be selected so that tooth enamel is not excessively abraded in normal use of the whitening dentifrice composition. Suitable abrasives include without limitation silica (in the form of silica gel, hydrated silica or precipitated silica), alumina, insoluble phosphates, calcium carbonate, resinous abrasives such as urea-formaldehyde condensation products and the like.

Among insoluble phosphates useful as abrasives are orthophosphates, polymetaphosphates and pyrophosphates. Illustrative examples are dicalcium orthophosphate dihydrate, calcium pyrophosphate, n-calcium pyrophosphate, tricalcium phosphate, calcium polymetaphosphate and insoluble sodium polymetaphosphate.

Average particle size of an abrasive, if present, is generally 0.1 to 30 μm for example, 1 to 20 μm or 5 to 15 μm. In some embodiments, one or more abrasives are present in an amount of 0.01 to 40 weight %, or 0.01 to 10 weight %, or such as 1.75 weight %, based on the total amount of the whitening dentifrice composition. In some embodiments, the abrasive is calcium pyrophosphate. In some embodiments, the calcium pyrophosphate is present in an amount from 5 to 50 weight %, such as 20 weight %, based on the total amount of the whitening dentifrice composition.

In various embodiments of the present disclosure, the whitening dentifrice composition includes an anticalculus agent. Suitable anticalculus agents include without limitation phosphates and polyphosphates (for example pyrophosphates), polyaminopropanesulfonic acid (AMPS), hexametaphosphate salts, zinc citrate trihydrate, polypeptides, polyolefin sulfonates, polyolefin phosphates, diphosphonates. In some embodiments, the anticalculus agent is present in an amount of 0.1 to 30 weight/o, based on the total amount of the whitening dentifrice composition. In some embodiments, the whitening dentifrice composition includes a mixture of anticalculus agents. In some embodiments, tetrasodium pyrophosphate (TSPP) and sodium tripolyphosphate (STPP) are used as the anticalculus agents. In some embodiments, the anticalculus agent includes 0.01 to 5% TSPP, such as 2 weight %, based on the total amount of the whitening dentifrice composition.

Another component of the present whitening dentifrice compositions may be a synthetic anionic polymeric polycarboxylate (SAPP), which acts as a stabilizer for the polyphosphate anti-tartar agent and which may help to block access of painful or pain-causing materials, such as sugars, to the tooth nerves.

In some embodiments, the whitening dentifrice composition optionally includes a source of fluoride ions. In some embodiments, the source of fluoride ions is selected from: fluoride, monofluorophosphate (MFP), and fluorosilicate salts. In some embodiments, one or more fluoride ion-releasing compounds are optionally present in an amount providing a total of 100 to 20,000 ppm, 200 to 5,000 ppm, or 500 to 2,500 ppm, fluoride ions. If present, the amount of fluoride in the present whitening dentifrice composition ranges from 0.1 to 1.1 weight %, typically 1.1 weight %, based on the total amount of the whitening dentifrice composition.

The whitening dentifrice compositions also may include a stannous ion or a stannous ion source to mitigate calcium loss. Suitable stannous ion sources include without limitation stannous fluoride, other stannous halides such as stannous chloride dihydrate, stannous pyrophosphate, organic stannous carboxylate salts such as stannous formate, acetate, gluconate, lactate, tartrate, oxalate, malonate and citrate, stannous ethylene glyoxide and the like. One or more stannous ion sources are optionally and illustratively present in a total amount of 0.01 to 10 weight %, for example 0.1 to 7 weight %, or 1 to 5 weight %, based on the total amount of the whitening dentifrice composition.

The whitening dentifrice compositions of the present disclosure may optionally include an antimicrobial (e.g., antibacterial) agent in addition to the zinc salts described herein. An illustrative list of useful antibacterial agents is provided in U.S. Pat. No. 5,776,435 to Gaffar et al., the contents of which are incorporated herein by reference. One or more antimicrobial agents in addition to the zinc salts described herein are optionally present in an antimicrobial effective total amount, typically 0.05 to 10 weight %, for example 0.1 to 3 weight %, based on the total amount of the whitening dentifrice composition.

Agents used to diminish teeth sensitivity such as potassium chloride, potassium nitrate, potassium citrate, dipotassium oxalate, or zinc phosphate may also be included in whitening dentifrice compositions of the present invention at concentrations of 0.1 to 10 weight %, based on the total amount of the whitening dentifrice composition.

In an aspect, the whitening dentifrice composition of the present disclosure provides a whitening benefit that is greater than that for a comparative dentifrice composition that is identical to the whitening dentifrice composition of the present disclosure, except that the comparative dentifrice composition has either a core shell silica with no zinc or has no core shell silica. The whitening benefit is at least one of a greater initial whitening benefit and a longer-lasting whitening benefit, as compared to a whitening benefit provided by a comparative dentifrice composition having an identical composition as that of the whitening dentifrice of the present disclosure, except that the comparative dentifrice composition either has core shell silica with no zinc or has no core shell silica.

In an embodiment, the initial whitening benefit is determined by at least one of initial whitening benefit determined by “Δb*ᵢₙᵢₜᵢₐₗ” value or “ΔWIOᵢₙᵢₜᵢₐₗ” value. The initial whitening benefit, “Δb*ᵢₙᵢₜᵢₐₗ” or “ΔWIOᵢₙᵢₜᵢₐₗ” can be determined by the difference between the whiteness index (WIO_{initial treatment}) or b*_{initial treatment} value of the tooth after 2 min of brushing with the product containing blue coloring agent and the whiteness index (WIO_{baseline}) or b*_{baseline} value of the tooth after removal of surface stains with the control silica toothpaste respectively, as shown below:

ΔWIOᵢₙᵢₜᵢₐₗ=WIO_{initial treatment}−WIO_{baseline}

Δb*ᵢₙᵢₜᵢₐₗ=b*_{after initial treatment}−b*_{baseline}

In another embodiment, the longer-lasting whitening benefit is determined by “Δb*_{longer-lasting}”, or “ΔWIO_{longer-lasting}”. The longer-lasting whitening benefit, “ΔWIO_{longer-lasting}” or “Δb*_{longer-lasting}” is the difference between the whiteness index (WIO_{after treatment & soaking}) or b*_{after treatment & soaking} value of the tooth after 2 min of brushing with the product containing blue coloring agent followed by soaking in saliva or artificial saliva for a duration of time such as 30 minutes, and the whiteness index (WIO_{baseline}) or b*_{baseline} value or of the tooth after removal of surface stains with the control silica toothpaste respectively, as shown below:

ΔWIO_{longer-lasting}=WIO_{after treatment & soaking}−WIO_{baseline}

Δb*_{longer-lasting}=b*_{after treatment & soaking}−b*_{baseline}

In an embodiment, the whitening benefit initial or longer-lasting provided by the whitening dentifrice composition of the present disclosure is at least 10%, or 20%, or 30% or 40%, or 50% or, 60% or 70% or 80% or 90% or 100/or more than 100%/o better than a comparative dentifrice composition that is identical to the whitening dentifrice composition of the present disclosure, except that the comparative dentifrice composition has either a core shell silica with no zinc or has no core shell silica.

Without wishing to be bound by theory, it is believed that the presence and amount of zinc ion in the whitening dentifrice composition of the present disclosure helps in the deposition of the blue coloring agent, as shown by the initial whitening benefit, whereas the presence and amount of Zn-CSS particles aids in the retention of the of blue coloring agent, as shown by the longer-lasting whitening benefit. Furthermore, the challenge associated with increasing the amount of zinc ion to deliver whitening benefit is on the taste profile of the finished whitening dentifrice composition. Surprisingly, it has been found that the impact of zinc ion to the taste profile is usually large but when associated with the Zn-CSS particle, it has very little perceived flavor change. Hence, the whitening dentifrice composition of the present disclosure provide a new way to increase the amount of zinc in the whitening dentifrice composition of the present disclosure by providing zinc ion as Zn-CSS particles, to deliver both the initial and the longer lasting whitening benefit, without any significant impact on the taste profile of the whitening dentifrice composition of the present disclosure.

### Methods/Uses

In various embodiments, the present disclosure provides methods to whiten an oral surface in a human or animal subject. The method may include contacting a tooth surface with the whitening dentifrice composition of the present disclosure. As used herein “animal subject” includes non-human mammals such as canines, felines and horses. The whitening dentifrice composition is contacted with an oral surface of the mammalian subject to thereby whiten teeth in a highly efficacious manner.

In various embodiments, the dentifrice composition prepared in accordance with the present disclosure may be applied regularly to an oral surface, for example on a daily basis, at least one time daily for multiple days, or alternately every second or third day. In some embodiments, the whitening dentifrice composition is applied to the oral surfaces from 1 to 3 times daily, for at least 2 weeks up to 8 weeks, from four months to three years, or more up to a lifetime.

In some embodiments, the whitening dentifrice composition, such as a gel, may be applied directly to the teeth using a delivery device, such as a pen, a liquid stick having an applicator, such as a felt tip, brush, roller ball, or non-woven pad, sufficient to effect whitening. In some embodiments, the whitening dentifrice composition of the present disclosure is maintained on the surface of the tooth for a plurality of minutes.

In some embodiments, the whitening dentifrice composition is maintained on the surface of a tooth for from 1 minute to 8 hours. In other embodiments, the whitening dentifrice composition is maintained on the surface of a tooth for from 5 minutes to 4 hours. In some embodiments, the whitening dentifrice composition is maintained on the surface of a tooth for from 10 minutes to 120 minutes. In some embodiments, the whitening dentifrice composition is maintained on the surface of a tooth for from 15 minutes to 60 minutes. In some embodiments, the whitening dentifrice composition is maintained on the surface of a tooth for from 20 minutes to 45 minutes.

Some embodiments provide a method where a delivery device, such as a whitening pen is stored within an oral care implement, such as a toothbrush. In some embodiments, the delivery device, such as a whitening pen is removed from the oral care implement prior to application of the whitening dentifrice composition to the tooth. In some embodiments, the whitening dentifrice composition is applied to the tooth after brushing with the oral care implement.

In one embodiment (embodiment 1), the disclosure provides for [1] the use of zinc core shell silica (Zn-CSS) particles as a tooth whitening enhancing agent in a whitening dentifrice composition, wherein the whitening dentifrice composition further comprises:

a) a blue coloring agent comprising at least one of a blue pigment and a blue dye, wherein the blue coloring agent has a blue to blue-violet color with a hue angle in the CIELAB system ranging from 200 degrees to 320 degrees; and

b) an orally acceptable vehicle comprising a non-aqueous solvent and water,

wherein the Zn-CSS particle comprises a silica core, and a surface of the silica core etched with a metal silicate, wherein the metal silicate is a silicate of zinc ion or zinc ion and a monovalent metal ion, and

wherein the whitening dentifrice composition is free from peroxide whitening agents and oxidants. Other embodiments are as follows:

[2] The use according to embodiment 1, wherein the zinc ion is present in an amount of from 0.01 to 1 weight %, based on the total amount of the whitening dentifrice composition.

[3] The use according to embodiment 1 or 2, wherein the surface of the silica core of the Zn-CSS particle comprises from 0.1 to 10 weight % of a metal silicate, based on the total amount of the whitening dentifrice composition.

[4] The use according to embodiment 1, 2, or 3, wherein the surface of the silica core of the Zn-CSS particle is represented by the following formula:

(SiO₂)ₚ[Oₒ*Mₙ⁺Znₘ²⁺Hₕ⁺].qH₂O

wherein O* is oxygen in the silicate form; M is a monovalent metal ion; Zn is divalent zinc ion; p, o, n, m, h and q are the atomic percentages of each component; and the total charge of each core shell silica particle is zero.

[5] The use according to any embodiment from 1 to 4, wherein the particles has a d(0.5) value of from 5 nm to 50 μm.

[6] The use according to any embodiment from 1 to 5, wherein the non-aqueous solvent is present in an amount from 25 to 95 weight % and water is present in an amount from 3 to 30 weight %, based on the total amount of the whitening dentifrice composition.

[7] The use according to any embodiment from 1 to 6, wherein the non-aqueous solvent is in present in an amount from 3 to 50 weight % and water is present in an amount from 25 to 70 weight %, based on the total amount of the whitening dentifrice composition.

[8] The use according to any embodiment from 1 to 7, wherein the blue coloring agent is a blue dye present in an amount of from 0.02 to 2 weight %, based on the total amount of the whitening dentifrice composition.

[9] The use according to any embodiment from 1 to 8, wherein the blue coloring agent comprises at least one of FD&C Blue#1, FD&C Blue #2, D&C Blue #4, CI Food Blue 5, and Acid Blue 1.

[10] The use according to any embodiment from 1 to 8, wherein the blue coloring agent is a blue pigment present in an amount of from 0.01 to 0.075 weight %, based on the total amount of the whitening dentifrice composition.

[11] The use according to any embodiment from 1 to 8 or 11, wherein the blue coloring agent comprises at least one of pigment violet 1 through to pigment violet 56 and pigment blue 1 through to pigment blue 83, as listed in the Color Index International.

[12] The use according to any embodiment from 1 to 8 or 11, wherein the blue coloring agent is a blue pigment #15 or a blue pigment #5.

[13] The use according to any embodiment from 1 to 12, wherein the whitening dentifrice composition provides a greater whitening benefit of the blue coloring agent than a comparative dentifrice composition that is identical to the whitening dentifrice composition of any embodiment from 1 to 12 except that the comparative dentifrice composition either has core shell silica with no zinc or has no core shell silica.

[14] The use according to any embodiment from 1 to 13, wherein the greater whitening benefit comprises at least one of greater initial whitening benefit and a longer-lasting whitening benefit.

[15] The use according to any embodiment from 1 to 14, wherein the non-aqueous solvent comprises glycerin, sorbitol, xylitol, propylene glycol, polyols, ketones, aldehydes, carboxylic acids or salts thereof, amines, or mixtures thereof.

[16] The use according to any embodiment from 1 to 16, further comprising one or more of an abrasive, a surfactant, a foaming agent, a vitamin, a fluoride ion source, an adhesive material, an enzyme, a humectant, a thickener, a sweetener, an anticalculus agent, a pH modifier, an antimicrobial agent, a preservative, and/or a flavoring.

[17] The use according to any embodiment from 1 to 16, wherein the whitening dentifrice composition is a gel.

[18] The use according to any embodiment from 1 to 17, wherein the whitening dentifrice composition is a toothpaste.

[19] A method for whitening a tooth surface comprising contacting the whitening dentifrice composition of any one of the preceding claims with the tooth surface

The examples and other embodiments described herein are exemplary and not intended to be limiting in describing the full scope of whitening dentifrice compositions and methods of this disclosure. Equivalent changes, modifications and variations of specific embodiments, materials, compositions and methods may be made within the scope of the present disclosure, with substantially similar results.

### EXAMPLES

### Materials Used

FD&C Blue #1 dye was obtained from Sensient Technologies Corporation. Blue Pigment #15 was obtained from BASF Corporation. Blue Pigment #15 used herein was used as is as free pigment and also as embedded in a polymer film in an amount of 16.7 weight %, based on the total amount of the polymer film and the pigment. The blue pigment #15 embedded in the polymer film was used at a 0.3 weight % level in the dentifrice composition to result in an overall amount of 0.05 weight % of blue pigment #15 in the dentifrice composition. High cleaning silica Zeodent® 105, regular silica like Zeodent® 114, and thickening silica like Zeodent® 165 were all obtained from by J. M. Huber. Core Shell Silica (CSS) and Zinc Core Shell Silica (Zn-CSS) were prepared as described below.

### Preparation of Core Shell Silica (CSS)

100 g of Silica (Zeodent® 105) was mixed with 150 g of water in a metal beaker fitted with overhead stirring. 12.5 g of 45% KOH was added to the rapidly mixing silica slurry. The slurry was mixed for 60 minutes. The mixture was neutralized with H₃PO₄. The CSS was isolated through vacuum filtration, washed with water, and dried in vacuum oven.

### Preparation of Zinc Core Shell Silica (Zn-CSS) Particles

100 g of Silica (Zeodent® 105) was mixed with 150 g water in a metal beaker fitted with overhead stirring. 12.5 g of 45% KOH was added to the rapidly mixing silica slurry. The slurry was mixed for 60 minutes. The mixture was neutralized with 10 g of ZnCl₂. The Zn-CSS particles were isolated through vacuum filtration, washed with water, and dried in vacuum oven. This results in 1 weight % of zinc ion in overall dentifrice composition

The total amount of zinc ion present on the Zn-CSS particle above is calculated to be 9 weight %. The total amount of zinc ion present on the Zn-CSS particle was adjusted by varying the amount of silica while keeping the amount of zinc chloride constant, as shown below:

| | | Amount of |
|---|---|---|
| | | Zn ion on |
| Silica | ZnCl₂ | the Zn-CSS |
| Added | Added | particle |
| (weight %) | (weight %) | (weight %) |
| 5 | 2 | 17 |
| 10 | 2 | 9 |
| 22 | 2 | 4 |
| 20 | 2 | 5 |

### Example 1: Whitening Efficacy of Non-Aaueous Composition Comprising Blue Dye as a Blue Coloring Agent

The efficacy of non-aqueous composition comprising blue dye as blue coloring agent and Zinc core shell silica was first tested in simple solutions, as shown in the Table 1 below.

The sample labeled “Dye Control” was a solution including 0.05 weight % blue dye, FD&C #1 in glycerin. The Dye Control solution did not include any form of zinc or core shell silica.

The comparative example labeled “Dye & CSS” was a solution including 0.05 weight % of blue dye, FD&C #1 and 10 weight % of core shell silica in glycerin.

The example labeled “Dye & Zn-CSS” was a solution including 0.05 weight % of blue dye, FD&C #1 and 10 weight % of zinc core shell silica in glycerin.

The formulations were evaluated for retention of blue dye on teeth from solution. The roots of human third molars were removed and the tooth was bisected from the crown through the root. Each half of the tooth was mounted in methacrylate resin and then secured in a brushing tray, enamel side facing out, using a thermal impression compound. Surface stains were removed from the teeth through brushing with a control silica toothpaste (10 min brushing with a 1:2 (w/w) slurry, 120 strokes/min). The teeth were rinsed with deionized water and cool air was used to remove excess water. The baseline color values were measured with the spectrophotometer (Spectroshade Micro, MHT technologies).

For the measurement of the blue retention for each formulation under test, 10 mL of the sample solution (formulations as shown in Table 1) was measured into the tray. The teeth were soaked for 2 min at room temperature. The solution was then rinsed with 100 mL of deionized water and cool air was used to remove excess water. The color values were measured with the spectrophotometer.

As used herein, the deposition of blue dye was quantified by Δb*, which is the change from yellow to blue with a negative value indicating a shift to blue.

As used herein, “Δb* initial” is the difference between the b* values of the tooth after surface stains are removed to that after 2 min soaking with the product containing a blue pigment.

A change in the whiteness index (WIO) was also calculated (with WIO being calculated as described in Joiner et al, “A review of tooth color and whiteness”, Journal of Dentistry 36S (2008), S2-S7), and shows that increased deposition of blue dye results in an increase in the whiteness of a tooth.

As used herein, “ΔWIO initial” is the change in whiteness of the tooth after 2 minutes of brushing with the product containing blue dye, as compared to the whiteness of the tooth after removal of surface stains with the control silica toothpaste (as described above).

The blue retention was also measured after soaking the above-treated teeth (i.e. brushed with the formulation under test for 2 mins) in artificial saliva. In this method, the above-treated teeth were soaked in 10 mL artificial saliva for 30 mins, then drained of saliva, and cool air was then used to remove excess water. The color values were again measured with the spectrophotometer. Δb* is the change in the yellow-blue axis in CIELAB color space. In the Tables 2 to 4, below, the value of Δb* reported is the difference between the b* value of the tooth after removal of surface stains by brushing for 10 minutes with the 1:2 (w/w) control silica toothpaste, and the b* value for the same tooth following 2 min brushing with the blue dye-containing test product (and—for “after 30 min soak”—subsequently followed by 30 minutes of soaking in artificial saliva). ΔWIO is the change in whiteness over the same period of time (and under the same treatment conditions) as for the Δb* measurements.

TABLE 1

| | | | | | Δb* | ΔWIO |
|---|---|---|---|---|---|---|
| | Non-AQ | | Δb* | ΔWIO | after 30 | after 30 |
| | Solution | Composition | initial | initial | min soak | min soak |
| Control 1 | Dye | 0.05 weight % FD&C #1 in | −1.9 | 7.63 | 0.4 | 0.24 |
| | Control | glycerin | | | | |
| Comparative | Dye & | 0.05 weight % FD&C #1 | −2.5 | 11.0 | −0.8 | 5.1 |
| Example A | CSS | and 10 weight % CSS in | | | | |
| | | glycerin | | | | |
| Example 1 | Dye & | 0.05 weight % FD&C #1 | −3.8 | 13.72 | −0.1 | 4.18 |
| | Zn-CSS | and 10 weight % Zn-CSS in | | | | |
| | | glycerin | | | | |

The above experiments showed that Zn-CSS was more effective than an unaltered CSS containing solution of dye in glycerin (Comparative Example A) in the delivery and was more effective than the control in retention of blue dye to teeth for an increase in tooth whitening. The greater initial whitening benefit as measured by Δb*ᵢₙᵢₜᵢₐₗ is 52% with respect to Comparative Example A and 100% with respect to Control. The greater initial whitening benefit as measured by ΔWIOᵢₙᵢₜᵢₐₗ is 25% with respect to Comparative Example A and 80% with respect to Control. Similarly, the greater longer lasting whitening benefit as measured by Δb*_{after 30 min} is 125% with respect to Control. The greater longer lasting whitening benefit as measured by ΔWIO after 30 min is 1642% with respect to Control.

### Example 2: Whitening Efficacy of Non-Aqueous Composition Comprising Blue Pigment as Blue Coloring Agent

The efficacy of non-aqueous composition comprising blue dye as blue coloring agent and Zinc core shell silica was first tested in simple solutions, using the procedure as described in Example 1 except that blue pigment was used instead of blue dye. The results are summarized in Table 2 below.

The sample labeled “Pigment Control” was a solution including 0.05% blue pigment in glycerin. The Pigment Control solution did not include any form of zinc or core shell silica.

The example labeled “Pigment & Zn-CSS” was a solution including 0.05 weight % of blue pigment #15 and 10 weight % of Zinc core shell silica in glycerin.

TABLE 2

| | | | | | Δb* | ΔWIO |
|---|---|---|---|---|---|---|
| | Non-AQ | | b* | ΔWIO | after 30 | after 30 |
| | Solution | Composition | initial | initial | min soak | min soak |
| Control 2 | Pigment | 0.05% blue pigment | −0.9 | 2.57 | −0.4 | 1.82 |
| | Control | #15 in glycerin | | | | |
| | | 0.05 weight % blue | | | | |
| Example 2 | Pigment + | pigment #15 and 10 | −1.3 | 4.44 | −0.6 | 2.17 |
| | Zn CSS | weight % Zn-CSS in | | | | |
| | | glycerin | | | | |

The above experiments showed that Zn-CSS was more effective than a solution of pigment in glycerin in the delivery and retention of blue pigment to teeth for an increase in tooth whitening. The greater initial whitening benefit as measured by Δb*ᵢₙᵢₜᵢₐₗ is 44% with respect to Control, and as measured by ΔWIOᵢₙᵢₜᵢₐₗ is 73% with respect to Control. Similarly, the greater longer lasting whitening benefit as measured by Δb*_{after 30 min} is 50% with respect to Control, and as measured by ΔWIO after 30 min is 19% with respect to Control. This is a surprising result, as the presence of Zn-CSS provides increased efficacy for both blue dye and blue pigment, even though blue dye and blue pigment have vastly different solubilities in glycerin, used here as the non-aqueous solvent.

### Example 3: Whitening Efficacy of Whitening Dentifrice Composition Comprising Blue Pigment and Zn-CSS Particles

### Step 3.1: Preparation of Whitening Dentifrice Composition Comprising Blue Pigment and Zn-CSS Particles

TABLE 3
Whitening Dentifrice Composition Comprising Blue Pigment

| | Target |
|---|---|
| Ingredient | Weight % |
| Glycerin | 10 |
| Polyethylene Glycol (PEG 600) | 3 |
| Sorbitol | 30 |
| Water | 19.4 |
| Zn Core Shell Silica (Zn-CSS) | 22 |
| delivering with 1 weight % of zinc | |
| ion | |
| Blue Pigment # 15 (from 0.3 | 0.05 |
| weight % of polymer film | |
| containing 16.7 weight % of | |
| pigment, with single layer film | |
| having a thickness of 2 mil) | |
| Thickener | 0.7 |
| Sodium Fluoride | 0.2 |
| Minors (aesthetic influencer, flavor, | Q.S |
| sweetener, thickener, surfactant, | |
| anticalculus agent) | |

Non-aqueous whitening dentifrice composition of Table 3 was made as follows:

1. Charged all non-aqueous solvents to a beaker. Added thickeners such as CMC and Xanthan gum and mixed until homogenous slurry was formed.

2. Mixed aqueous solvents with fluoride and sweetener until homogenous solution was obtained. Added solution to the homogeneous slurry. Added free pigment as colorant here or if pigment is embedded, then added at the end.

3. Added Zn-CSS particles and mixed under vacuum. Added surfactants, flavor, and aesthetic influencers. Added pigment as embedded in a polymer film. Pigment #15 used herein was embedded in an amount of 16.7 weight % in a polymer film, based on the total amount of the polymer film and the pigment. Pigment #15 embedded in the polymeric film was used at a 0.3 weight % to result in an overall amount of 0.05 weight % of Pigment 5 in the dentifrice composition.

### Step 3.2: Evaluation of Whitening Efficacy of Whitening Dentifrice Compositions Comprising Blue Pigment and Zn-CSS Particles

The whitening effect of dentifrice compositions were evaluated by monitoring the retention of blue pigment blue #15 on teeth and resulting tooth whiteness as follows:

The roots of human third molars were removed and the tooth was bisected from the crown through the root. Each half of the tooth was mounted in methacrylate resin and then secured in a brushing tray, enamel side facing out, using a thermal impression compound. Four teeth were mounted per tray. The teeth were brushed for 10 minutes with a 1:2 (w/w) silica toothpaste slurry to remove any extrinsic surface stains, rinsed with deionized water, dried with cool air, and baseline CIELAB measurements were recorded with a spectrophotometer (Spectroshade Micro. MHT technologies). The teeth were submerged in saliva (9 mL/tray), aged at 37° C. with gentle agitation for 15 minutes. The test toothpaste (6 g) was then added to the tray already containing 9 mL saliva, the teeth were brushed for 2 minutes, rinsed with 100 mL deionized water, dried with cool air, and CIELAB measurements were recorded. The teeth were aged in saliva for 10 minutes and 30 minutes in saliva. Teeth were rinsed and dried with cool air before CIELAB measurements were recorded at each time during the soaking cycle. The results of the measurement are summarized in the Table 4 below.

Table 4 shows the whitening effect of two dentifrice compositions, Control 3 and Example 3. Example 3 corresponds to the composition of Table 3 and includes Zn Core Shell Silica (Zn-CSS) and blue pigment, whereas Control 3 corresponds to the composition of Table 3 without Zn-CSS. Both compositions contain 0.05% Blue Pigment #15 and were prepared the same way except the presence or absence of Zn-CSS.

Both formulations shown in Table 4: Control and Example 3 contain 0.05% Pigment #15 coming from 0.3 weight % of polymer film containing 16.7 weight/o of Pigment #15, with single layer film having a thickness of 2 mil (50.8 micrometer).

TABLE 4
Whitening Efficacy

| | | Δb* | | | ΔWIO | | |
|---|---|---|---|---|---|---|---|
| | Composition shown | | 10 min | 30 min | | 10 min | 30 min |
| | in Table 3 | Initial | saliva | saliva | Initial | saliva | saliva |
| Control 3 | 0% Zn CSS | −3.1 | −2.7 | −2.3 | 9 | 8 | 7 |
| Example 3 | 22 weight % Zn-CSS | −3.4 | −3.6 | −3.3 | 11 | 11 | 11 |
| | delivering 1 weight | | | | | | |
| | % of zinc ion | | | | | | |

The data in Table 4 demonstrate that the addition of Zn-CSS to the dentifrice composition resulted in increased deposition of blue pigment and an increase in the retention time of the pigment on teeth. The greater initial whitening benefit as measured by Δb*ᵢₙᵢₜᵢₐₗ is 10% with respect to Control, and as measured by ΔWIOᵢₙᵢₜᵢₐₗ is 22% with respect to Control. Similarly, the greater longer lasting whitening benefit as measured by Δb*_{after 30 min} is 43% with respect to Control, and as measured by ΔWIO after 30 min is 57% with respect to Control.

### Example 4: Preparation of Whitening Dentifrice Composition Comprising Blue Dye, Zn-CSS Particles

The same procedures as used in Step 3.1 of Example 3 were used to make a whitening dentifrice composition, except that FD&C Blue #1 dye was used instead of pigment blue #15, as shown below in Table 5.

TABLE 5
Whitening dentifrice composition comprising blue dye and
low-water content orally acceptable vehicle

| | Target Weight %, |
|---|---|
| | based on the total |
| | amount of the |
| Ingredient | Dentifrice composition |
| Glycerin | 58.857 |
| Polyethylene Glycol | 3 |
| Water | 7 |
| Zn Core Shell Silica (Zn-CSS) | 22 |
| delivering with 1 weight % of zinc | |
| ion | |
| FD&C Blue #1 | 0.05 |
| Thickener | 0.7 |
| Sodium Fluoride | 0.2 |
| Minors (aesthetic influencer, | Q.S |
| flavor, sweetener, thickener, | |
| surfactant, anticalculus agent) | |

What is claimed is:

1. A whitening dentifrice composition comprising:
a blue coloring agent comprising at least one of a blue pigment and a blue dye, wherein the blue coloring agent has a blue to blue-violet color with a hue angle in the CIELAB system ranging from 200 degrees to 320 degrees;
a zinc core shell silica (Zn-CSS) particle, wherein the Zn-CSS particle comprises a silica core, and a surface of the silica core etched with a metal silicate, wherein the metal silicate is a silicate of zinc ion and optionally a monovalent metal ion; and
an orally acceptable vehicle comprising a non-aqueous solvent and water, wherein the whitening dentifrice composition is free from peroxide whitening agents and oxidants.

2. The whitening dentifrice composition of claim 1, wherein the zinc ion is present in an amount of from 0.01 to 1 weight %, based on the total amount of the whitening dentifrice composition.

3. The whitening dentifrice composition of claim 1, wherein the surface of the silica core of the Zn-CSS particle is represented by the following formula:
(SiO₂)ₚ[Oₒ*Mₙ⁺Znₘ²⁺Hₕ⁺].qH₂O
wherein O* is oxygen in the silicate form; M is a monovalent metal ion; Zn is divalent zinc ion; p, o, n, m, h and q are the atomic percentages of each component; and the total charge of each core shell silica particle is zero.

4. The whitening dentifrice composition of claim 1, wherein the non-aqueous solvent is present in an amount from 25 to 95 weight % and water is present in an amount from 3 to 30 weight %, based on the total amount of the whitening dentifrice composition.

5. The whitening dentifrice composition of claim 1, wherein the non-aqueous solvent is in present in an amount from 3 to 50 weight % and water is present in an amount from 25 to 70 weight %, based on the total amount of the whitening dentifrice composition.

6. The whitening dentifrice composition of claim 1, wherein the blue coloring agent is a blue dye present in an amount of from 0.02 to 2 weight %, based on the total amount of the whitening dentifrice composition.

7. The whitening dentifrice composition of claim 1, wherein the blue coloring agent comprises at least one of FD&C Blue#1, FD&C Blue #2, D&C Blue #4, CI Food Blue 5, and Acid Blue 1.

8. The whitening dentifrice composition of claim 1, wherein the blue coloring agent is a blue pigment present in an amount of from 0.01 to 0.075 weight %, based on the total amount of the whitening dentifrice composition.

9. The whitening dentifrice composition of claim 1, wherein the blue coloring agent comprises at least one of a violet pigment #1 through to violet pigment #56 and pigment blue 1 through to pigment blue 83, as listed in the Color Index International.

10. The whitening dentifrice composition of claim 1, wherein the blue coloring agent is a blue pigment #15 or a blue pigment #5.

11. The whitening dentifrice composition of claim 1, wherein the whitening dentifrice composition provides a greater whitening benefit for the blue coloring agent than a comparative dentifrice composition that is identical to the whitening dentifrice composition of claim 1, except that the comparative dentifrice composition either has core shell silica with no zinc (CSS) or has no core shell silica.

12. The whitening dentifrice composition of claim 11, wherein the greater whitening benefit comprises at least one of greater initial whitening benefit and a longer-lasting whitening benefit.

13. The whitening dentifrice composition of claim 1, wherein the non-aqueous solvent comprises glycerin, sorbitol, xylitol, propylene glycol, polyols, ketones, aldehydes, carboxylic acids or salts thereof, amines, or mixtures thereof.

14. A method for whitening a tooth surface comprising contacting the whitening dentifrice composition of claim 1 with the tooth surface.

15. A use of a zinc core shell silica (Zn-CSS) particle as a tooth whitening enhancing agent in a whitening dentifrice composition, wherein the whitening dentifrice composition further comprises:
a blue coloring agent comprising at least one of a blue pigment and a blue dye, wherein the blue coloring agent has a blue to blue-violet color with a hue angle in the CIELAB system ranging from 200 degrees to 320 degrees; and
an orally acceptable vehicle comprising a non-aqueous solvent and water,
wherein the Zn-CSS particle comprises a silica core, and a surface of the silica core etched with a metal silicate, wherein the metal silicate is a silicate of Zinc ion or Zinc ion and a monovalent metal ion, and
wherein the whitening dentifrice composition is free from peroxide whitening agents and oxidants.
